(19) United States
(12) Patent Application Publication
(10) Pub. No.: US 2016/0106568 A1
(43) Pub. Date: Apr. 21, 2016

(54) SPINE CORRECTION AND PELVIS STRENGTHENING APPARATUS

(71) Applicant: NEULBEET CO., LTD., YANGJU-SI (KR)

(72) Inventor: Jang Hwan LEE, Uijeongbu-si (KR)

(21) Appl. No.: 14/549,282

(22) Filed: Nov. 20, 2014

(30) Foreign Application Priority Data
Oct. 15, 2014 (KR) 10-2014-0138882

(51) Int. Cl. A61F 5/37

(52) U.S. Cl. CPC A61F 5/3715

(57) ABSTRACT

A spine correction and pelvis strengthening apparatus is provided. The spine correction and pelvis strengthening apparatus includes: a fixed securing means which includes: a first knee splaying prevention plate downwardly bent from one side of the fixed securing means and shaped like a hand; a first horizontal plate extending from the first knees splaying prevention plate and having a guide recess formed on a bottom surface of the first horizontal plate; and a securing bracket formed at the other side of the fixed securing means and extending from the first horizontal plate, the first knees splaying prevention plate, the first horizontal plate, and the securing bracket being integrally formed with one another; and a variable securing means which includes: a second knees splaying prevention plate downwardly bent from one side of the variable securing means and shaped like a hand; a second horizontal plate extending from the second knees splaying prevention plate; and a variable extension bar formed at the other side of the variable securing means, the second knees splaying prevention plate, the second horizontal plate, and the variable extension bar being integrally formed with one another.

### CROSS-REFERENCE TO RELATED APPLICATION(S) AND CLAIM OF PRIORITY

The present application claims the benefit under 35 U.S.C. §119(a) to a Korean patent application filed in the Korean Intellectual Property Office on Oct. 15, 2014, and assigned Serial No. 10-2014-0138882, the entire disclosure of which is hereby incorporated by reference.

### TECHNICAL FIELD OF THE INVENTION

The present invention relates generally to a spine correction and pelvis strengthening apparatus, which can straighten or correct user's spine by keeping user's knees together when a user sits on a chair, can improve blood circulation by preventing dislocation of a hip joint, strengthening the pelvis, and correcting pelvic torsion, and thus can keep user's balance and is beneficial to user's health.

### BACKGROUND OF THE INVENTION

Modern people spend much time sitting on chairs. When people sit on chairs, their legs are naturally splayed out due to a human body structure in which spine, pelvis and legs are jointed with one another.

When the legs are splayed out, people's postures lean forward. Thus, the waist and the neck bend forward along the spine. Office workers or students who spend much time sitting in such a posture are likely to suffer from deformity in posture.

That is, sitting with legs apart may cause dislocation of a hip joint, pelvic torsion, scoliosis, spinal stenosis, and the like, and may cause bad blood circulation.

Specifically, when the knees are splayed out, the weight is shifted to one side and thus the waist is crooked. Therefore, the upper body leans forward and spinal deformity becomes more serious.

In addition, women who wear skirts are conscious of the eyes of others and thus tend to sit with knees together. When a woman takes a long trip by using public transportation, sitting with knees together for a long time is not an easy work. Therefore, the woman may unconsciously splay her legs out and thus not only she but other people may be embarrassed.

As described above, most of the modern people unconsciously splay their legs out when sitting on chairs even when they recognize the physical problem and other problems, and considerable effort and much time are required to artificially correct this posture. When people try to keep their knees together consciously within a short time, a force is exerted to the entire legs and the pelvis, causing fatigue and disturbing concentration.

Therefore, there is a need for a means for keeping legs together to prevent physical deformity and keep women's modest attitudes.

Korean Utility Model Registration No. 20-0423094 titled “A band for keeping knees together”, which is a related-art method to solve the above-described problems, disclose the technical matters in the following claims:

Claim 1

A band for keeping knees together comprising: a banding means which is worn around knees of both legs to prevent knees from being splayed out; connecting means which is coupled to opposite ends of the banding means and makes the banding means form a looped curve though mutual connection; and a length changing means which is provided on the banding means to change a length of the banding means.

Claim 2

The band for keeping knees together of claim 1, wherein the banding means is made of a fabric band having passing holes formed thereon, wherein the connecting means comprises a protrusion elastic body formed at one side, and a securing instrument formed at the other side and having a recess that the protrusion elastic body is inserted into and elastically secured with, and wherein the length changing means comprises three pins placed in a horizontal position and opposite ends of the length changing means comprise clips connected to each other.

The above-described related-art method has no problem in keeping the knees together forcedly. However, in the case of office workers, they frequently rise from their chairs to respond to a call from their superior or to do other works. That is, the office workers repeat sitting on a chair and rising from the chair many times. In this situation, it is inconvenient to wear or take off the band and thus it is difficult to use it.

That is, a user should remove the protrusion elastic body of the connecting means from the securing instrument to rise from his/her chair, which is very cumbersome and causes inefficiency.

### PRIOR ART DOCUMENTS

### Patent Documents

1. Korean Utility Model Registration No. 0423094

2. Korean Utility Model Registration No. 0383745

3. Korean Utility Model Registration No. 0275189

4. Korean Patent Application No. 10-2009-2627

### SUMMARY OF THE INVENTION

To address the above-discussed deficiencies of the prior art, it is a primary aspect of the present invention to improve convenience of wearing/taking off and speed by focusing on general office workers who frequently rise from their chairs and sit on the chairs.

Another object of the present invention is to correct user's spine by keeping the spine straight and thus preserver user's health of internal organs or urinary system, and to improve blood circulation by providing a structure for placing user's pelvis in a correct position.

Another object of the present invention is to strengthen a sphincter muscle by keeping user's knees together when the user sits on a chair and thus always tightening the sphincter muscle, strengthen thighs, and furthermore prevent lower body obesity.

According to one aspect of the present invention, a spine correction and pelvis strengthening apparatus includes: a fixed securing means which includes: a first knee splaying prevention plate downwardly bent from one side of the fixed securing means and shaped like a hand; a first horizontal plate extending from the first knees splaying prevention plate and having a guide recess formed on a bottom surface of the first horizontal plate; and a securing bracket formed at the other side of the fixed securing means and extending from the first horizontal plate, the first knees splaying prevention plate, the first horizontal plate, and the securing bracket being integrally formed with one another; and a variable securing means which includes: a second knees splaying prevention plate downwardly bent from one side of the variable securing means and shaped like a hand; a second horizontal plate extending from the second knees splaying prevention plate; and a variable extension bar formed at the other side of the variable securing means, the second knees splaying prevention plate, the second horizontal plate, and the variable extension bar being integrally formed with one another.

The first knees splaying prevention plate and the second knees splaying prevention plate may be bowed inwards.

The variable extension bar may include a guide recess formed on opposite sides and a locking portion which is saw-toothed.

The securing bracket may include: a leading end guider integrally formed with a leading end of the securing bracket; a guide recess formed along opposite sides toward the rear end in a lengthwise direction in a cutaway state to be consistent with a width of the guide recess; a cutaway recess formed between the guide recess and the leading end guider, a tension fixing portion formed at opposite sides of the securing bracket, and a hooking portion integrally formed with an end of the tension fixing portion and located in the cutaway recess. The hooking portion may be operated according to an operation of the tension fixing portion and an end of the hooking portion may be engaged with or disengaged from the locking portion of the variable extension bar.

According to the present invention, by banding legs or keeping legs together, the pelvis is not curved and the spine is kept straight. Therefore, the flow of energy from head to toe can be improved and a detoxifying function of a blood flow can be achieved. Therefore, the body's metabolism can be activated and thus fatigue can be relieved. In addition, taking a good posture everyday is very helpful to blood detoxification.

In addition, according to the present invention, the spine correction and pelvis strengthening apparatus can improve convenience of wearing/taking off and speed by focusing on general office workers who frequently rise from chairs and sit on chairs, and thus can be easily used in offices. In addition, the spine correction and pelvis strengthening apparatus can always keep user's knees together and thus can tighten a user's sphincter muscle, so that the sphincter muscle and the thighs can be strengthened and furthermore lower body obesity can be prevented.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a perspective view of a spine correction and pelvis strengthening apparatus according to the present invention;

FIG. 2 is a perspective view of a fixed securing means in the spine correction and pelvis strengthening apparatus according to the present invention;

FIG. 3 is a perspective view a variable securing means in the spine correction and pelvis strengthening apparatus according to the present invention;

FIG. 4 is an exploded perspective view of the spine correction and pelvis strengthening apparatus according to the preset invention; and

FIG. 5 is a view of the spine correction and pelvis strengthening apparatus when it is in use.

### DETAILED DESCRIPTION OF THE INVENTION

Reference will now be made in detail to the embodiment of the present general inventive concept, examples of which are illustrated in the accompanying drawings, wherein like reference numerals refer to the like elements throughout. The embodiment is described below in order to explain the present general inventive concept by referring to the drawings.

Hereinafter, a detailed configuration and an operation of a spine correction and pelvis strengthening apparatus according to the present invention will be explained with reference to the accompanying drawings.

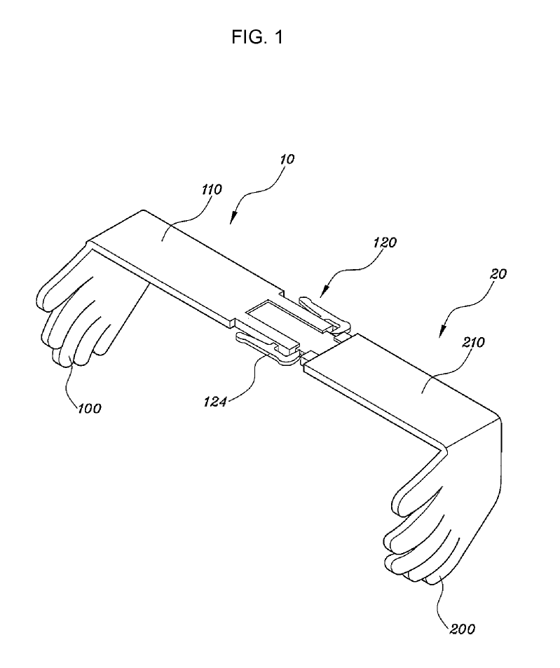

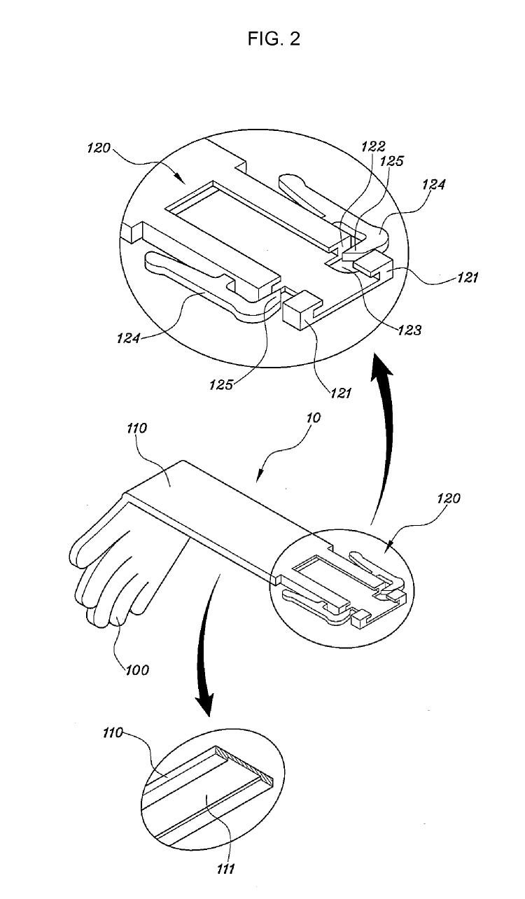

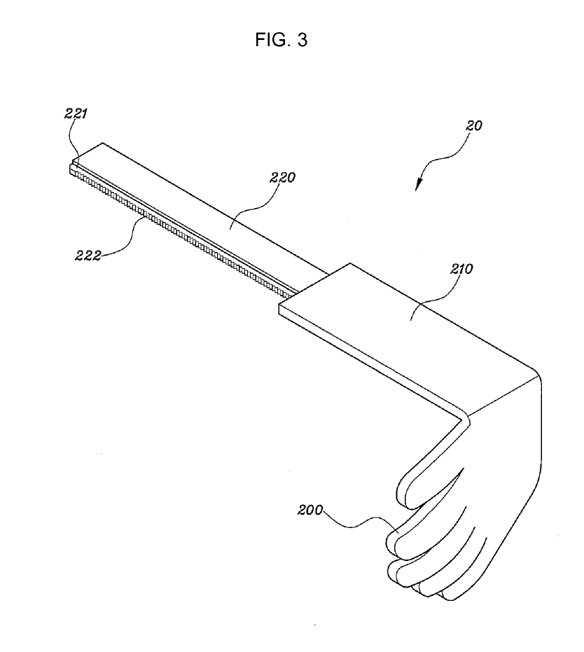

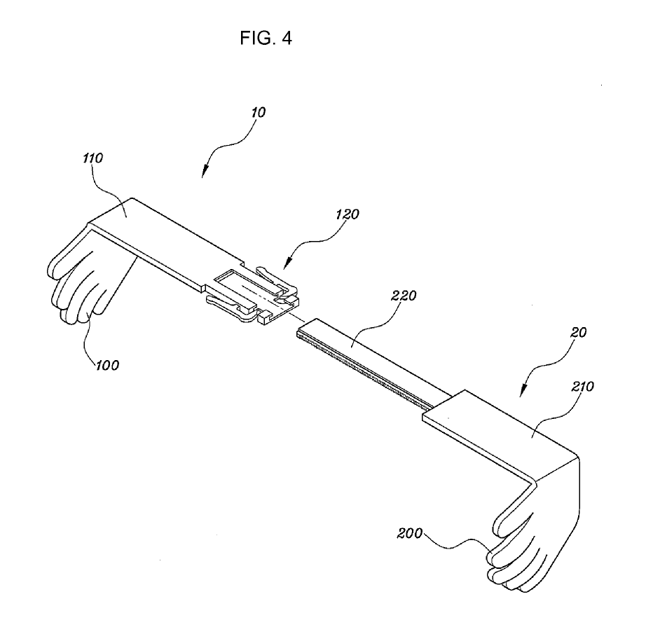

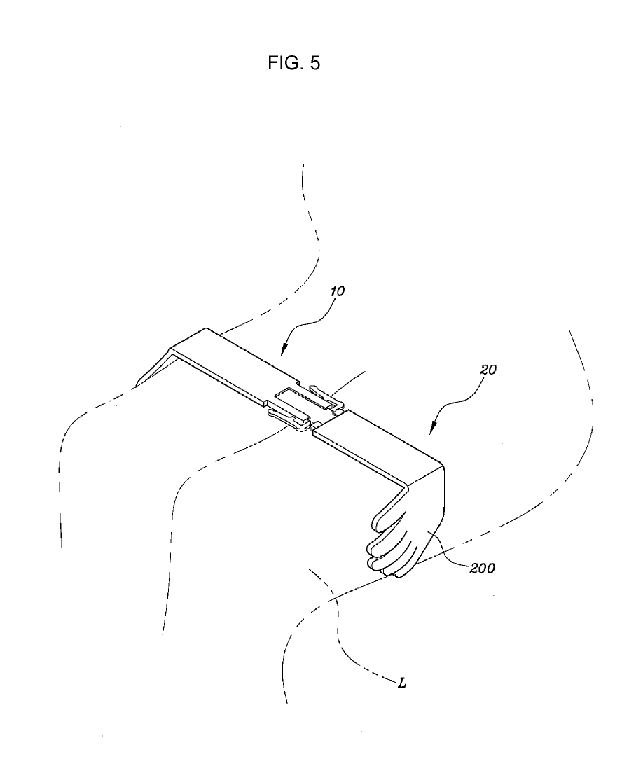

FIG. 1 is a perspective view of a spine correction and pelvis strengthening apparatus according to the present invention, FIG. 2 is a perspective view of a fixed securing means in the spine correction and pelvis strengthening apparatus according to the present invention, FIG. 3 is a perspective view a variable securing means in the spine correction and pelvis strengthening apparatus according to the present invention, FIG. 4 is an exploded perspective view of the spine correction and pelvis strengthening apparatus according to the preset invention, and FIG. 5 is a view of the spine correction and pelvis strengthening apparatus when it is in use.

The present invention is generally divided into a fixed securing means 10 and a variable securing means 20.

As shown in FIG. 2, the fixed securing means 10 includes a first knee splaying prevention plate 100 downwardly bent from one side of the fixed securing means 10 and shaped like a hand, a first horizontal plate 110 extending from the first knees splaying prevention plate 100 and having a guide recess 111 formed on a bottom surface of the first horizontal plate 110, and a securing bracket 120 formed at the other side of the fixed securing means 10 and extending from the first horizontal plate 110. The first knees splaying prevention plate 100, the first horizontal plate 110, and the securing bracket 120 are integrally formed with one another.

As shown in FIG. 3, the variable securing means 20 includes a second knees splaying prevention plate 200 downwardly bent from one side of the variable securing means 20 and shaped like a hand, a second horizontal plate 210 extending from the second knees splaying prevention plate 200, and a variable extension bar 220 formed at the other side of the variable securing means 20. The second knees splaying prevention plate 200, the second horizontal plate 210, and the variable extension bar 220 are integrally formed with one another.

The first knees splaying prevention plate 100 and the second knees splaying prevention plate 200 are bowed inwards.

The variable extension bar 220 includes a guide recess 221 formed on opposite sides and a locking portion 222 which is saw-toothed.

The securing bracket 120 includes a leading end guider 121 integrally formed with a leading end of the securing bracket 120, a guide recess 122 formed along opposite sides toward the rear end in a lengthwise direction in a cutaway state to be consistent with a width of the guide recess 111, a cutaway recess 123 formed between the guide recess 122 and the leading end guider 121, a tension fixing portion 124 formed at opposite sides of the securing bracket 120, and a hooking portion 125 integrally formed with an end of the tension fixing portion 124 and located in the cutaway recess 123. The hooking portion 125 is operated according to the operation of the tension fixing portion 124 and the end of the hooking portion 125 is engaged with or disengaged from the locking portion 222 of the variable extension bar 220.

Hereinafter, an operation of the present invention will be explained.

According to the present invention, first, the variable extension bar 220 of the variable securing means 20 is inserted into the leading end guider 121 of the fixed securing means 10 and is slid along the guide recess 111 formed on the bottom surface of the first horizontal plate 110, as shown in FIG. 4. Then, the fixed securing means 10 and the variable securing means 20 are connected with each other as shown in FIG. 1.

In this state, in order to fit the spine correction and pelvis strengthening apparatus over user's knees or thighs (L) as shown in FIG. 5, the rear end of the tension fixing portion 124 of the securing bracket 120 is pressed. Then, the hooking portion 125 is disengaged from the locking portion 222. In this state, the user determines an appropriate position by horizontally sliding the variable extension bar 220 along the guide recess 111 up to a desired length. Then, when the user releases the pressure to the hooking portion 125 of the tension fixing portion 124, the hooking portion 125 is engaged with the locking portion 222.

When the user rises from his/her seat, the user releases the hooking portion 125 from the locking portion 222 by pressing the rear end of the tension fixing portion 124 and removes the spine correction and pelvis strengthening apparatus from his/her knees or thighs. However, in an urgent case, the user can easily remove the spine correction and pelvis strengthening apparatus simply by pulling up the first horizontal plate 110 or the second horizontal plate 210.

According to the present invention, the spine correction and pelvis strengthening apparatus can improve convenience of wearing/taking off and speed by focusing on general office workers who frequently rise from chairs and sit on chairs, and thus can be easily used in offices. In addition, the spine correction and pelvis strengthening apparatus can always keep user's knees together and thus can tighten a user's sphincter muscle, so that the sphincter muscle and the thighs can be strengthened and furthermore lower body obesity can be prevented.

By sitting with knees together, the pelvis is not curved and the spine is kept straight. Therefore, the flow of energy from head to toe can be improved and a detoxifying function of a blood flow can be achieved. Therefore, the body's metabolism can be activated and thus fatigue can be relieved. In addition, taking a good posture everyday is very helpful to blood detoxification.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A spine correction and pelvis strengthening apparatus comprising:
a fixed securing means which comprises: a first knee splaying prevention plate downwardly bent from one side of the fixed securing means and shaped like a hand; a first horizontal plate extending from the first knees splaying prevention plate and having a guide recess formed on a bottom surface of the first horizontal plate; and a securing bracket formed at the other side of the fixed securing means and extending from the first horizontal plate, the first knees splaying prevention plate, the first horizontal plate, and the securing bracket being integrally formed with one another; and
a variable securing means which comprises: a second knees splaying prevention plate downwardly bent from one side of the variable securing means and shaped like a hand; a second horizontal plate extending from the second knees splaying prevention plate; and a variable extension bar formed at the other side of the variable securing means, the second knees splaying prevention plate, the second horizontal plate, and the variable extension bar being integrally formed with one another.

2. The spine correction and pelvis strengthening apparatus of claim 1, wherein the first knees splaying prevention plate and the second knees splaying prevention plate are bowed inwards.

3. The spine correction and pelvis strengthening apparatus of claim 1, wherein the variable extension bar comprises a guide recess formed on opposite sides and a locking portion which is saw-toothed.

4. The spine correction and pelvis strengthening apparatus of claim 1, wherein the securing bracket comprises: a leading end guider integrally formed with a leading end of the securing bracket; a guide recess formed along opposite sides toward the rear end in a lengthwise direction in a cutaway state to be consistent with a width of the guide recess; a cutaway recess formed between the guide recess and the leading end guider, a tension fixing portion formed at opposite sides of the securing bracket, and a hooking portion integrally formed with an end of the tension fixing portion and located in the cutaway recess, and
wherein the hooking portion is operated according to an operation of the tension fixing portion and an end of the hooking portion is engaged with or disengaged from the locking portion of the variable extension bar.
